(19) United States
(12) Patent Application Publication
(10) Pub. No.: US 2011/0092589 A1
(43) Pub. Date: Apr. 21, 2011

(54) Treatment of Inflammatory Conditions and Diseases with Metal-Thiols

(75) Inventors: Dhanonjoy Saha, Matthews, NC (US); Philip Domenico, New York, NY (US)

(73) Assignee: THE CHARLOTTE-MECKLENBURG HOSPOTAL AUTHORITY, Charlotte, NC (US)

(21) Appl. No.: 12/920,787

(22) PCT Filed: Mar. 10, 2009
(86) PCT No.: PCT/US2009/036609

§ 371 Date: Dec. 13, 2010

Related U.S. Application Data
(60) Provisional application No. 61/068,776, filed on Mar. 10, 2008.

(51) Int. Cl. A61K 31/29; A61P 29/00; A61P 17/06; A61P 11/06; A61P 19/02; A61P 1/00; A61P 25/28; A61P 25/16; A61P 25/06; A61P 25/08

(52) U.S. Cl. 514/503

(57) ABSTRACT

The present invention provides a method for the treatment of a disease or disorder related to an inflammatory condition comprising administering to a patient in need thereof a therapeutically effective amount of a pharmaceutical composition comprising a metal and a thiol. In particular, embodiments of the present invention are directed towards methods of treating inflammatory conditions that are associated with elevated levels of free TNFα. In one embodiment, the present invention can be used to treat and/or ameliorate the effects of a wide variety of diseases or disorders related to an inflammatory condition including one or more of the following inflammation related diseases/disorders: arthritis, rheumatoid arthritis, asthma, psoriasis, systemic lupus erythematosus, inflammatory bowel syndrome, chronic obstructive respiratory diseases (COPD), fibromyalgia and the neurological diseases and disorders multiple sclerosis, Alzheimer's disease, Parkinson's disease, Huntington's disease, vascular dementia, epilepsy, migraines, stroke and trauma.

### FIELD OF THE INVENTION

The present invention relates to a method of treating inflammatory diseases with metal thiols, and in particular to a method of treating chronic inflammatory diseases with an anti-inflammatory agent comprising a metal-thiol.

### BACKGROUND OF THE INVENTION

Inflammation is an important aspect of the natural defense process. Inflammation becomes a pathological process, requiring medical intervention, when inflammatory mediators cause excessive damage to the surrounding tissue. Examples of such pathological processes are rheumatoid arthritis (RA) and psoriasis. Recently, a significant inflammatory component has been found in other types of disease, for example, neurological disorders such as multiple sclerosis and Alzheimer's disease. A common feature of many inflammatory diseases is an over-activation of pro-inflammatory cytokines, such as Tumor Necrosis Factor-alpha (TNF-α). In particular, inflammation can be associated and lead to chronic inflammatory diseases (CID). The process of inflammation is virtually the same in different diseases (Schwartz et al. 2006). A wide variety of these diseases are linked to autoimmunity, genetic factors, allergy, neurodegeneration, or impaired host response to infections (Schwartz et al. 2006; Pincus 2005). These immune-mediated inflammatory diseases target many parts of the human body including nervous system, gastrointestinal system, skin and other connective tissues and are associated with considerable morbidity and early mortality (Schwartz et al. 2006; Pincus 2005).

Cytokines are proteins naturally produced by immune cells and trigger responses to toxins, allergens, injury and microbial pathogens (Pincus 2005). Malfunction of these proteins can result in tissue damage and disease. The pathology underlying CID is the over-activation of pro-inflammatory cytokines such as TNF-α, Interleukin-1 (IL-1), IL-6, and IL-8.

Cytokines also act as chemical messengers between cells to regulate physiologic processes, such as cell growth, differentiation, inflammation, repair, and immunity. More than 150 cytokines have been identified. One such cytokine is tumor necrosis factor (TNF). TNF is an important pro-inflammatory cytokine produced by a variety of immune cells but is also rapidly induced in many non-immune cells by a variety of stimuli. TNF is normally present at low concentrations, where it plays a role in tumor surveillance, inflammatory response regulation, and local tissue repair. TNF has the potential to affect many organs and it has been implicated in both acute and chronic inflammatory conditions.

TNFα is released in large amounts in response to lipopolysaccharide (LPS). An acute phase response occurs a few hours after sensitization of mice with a few micrograms of LPS, resulting in fever, disrupted sleep, and leukocytosis. A large LPS stimulus promotes an overwhelming inflammatory response, leading to multiple organ dysfunction, septic shock, and death. In blood vessels and smooth muscle, TNFα induces a procoagulant state. In synovium, it promotes the upregulation of adhesion molecules, with an influx of inflammatory cells. In the central nervous system it disregulates pathways, causing fever and sleep disruption. In bone, it promotes destruction and repair. TNFα also stimulates repair, scar formation, and the lysis of tumor cells.

TNF is normally expressed in cell membranes as a homotrimer, with aggregated monomers. These are cleaved by the metalloproteinase (MP), TNF-alpha converting enzyme (TACE), allowing TNFα molecules to circulate freely and to bind to TNF receptors on numerous target cells. TACE is a zinc-dependent disintegrin and MP of the metzincin superfamily. It helps regulate shedding of an array of membrane-anchored, bioactive molecules such as cytokines, growth factors and receptors. Notable examples include pro-TNF-α and fractalkine, receptors such as Notch, TNFα, R I, II and molecules associated with cell adhesion, such as L-selectin. TACE is the protease responsible for the release of soluble, free TNFα from its parent membrane bound pro-TNFα. TNF α then mediates pro-inflammatory responses from other cytokines, generating free radicals and oxidative damage. TNF has also been linked to chronic inflammation like rheumatoid arthritis (RA), with pathogenesis involving bone and cartilage destruction. Other diseases were also associated with this are psoriasis, ankylosis spondylitis, bursitis, tendonitis, and chronic obstructive pulmonary disease (COPD). Systemic inflammation is linked to an argosy of disorders, from heart disease to depression, often accompanied by elevated C-reactive protein (CRP) levels and TNF-mediated events.

Traditional anti-inflammatory drugs include steroids which suppress inflammation at multiple sites and cause systemic side effects. More modern drugs, called biological agents (biologics), target cytokines more locally and include: TNF-a blockers (e.g. infliximab, etanercept, and adalimumab), IL-1 blockers (e.g. anakinra), and 11-6 blockers (e.g. tocilizumab). Biologic therapies avoid the multiorgan toxicity often seen with traditional immunosuppressive drugs such as methotrexate and cyclosporine. For example, TNF-α blockers have demonstrated significant clinical improvement in patients with diseases such as RA.

One concern about biological agents as drugs is that they are proteins. Proteins are poor drugs especially for chronic diseases. For example, proteins are not effective when taken orally and therefore should be administered by injection. However, injection can cause allergic reactions. Other side effects might include increase in the risk of infection and malignancy. Finally, these medications can be prohibitively expensive ranging from $3,000 to $10,000 per year. Another risk is the development of antibodies to the biological agents over time. The immuno-suppressive drugs, methotrexate and cyclosporine, have been used as systemic therapies for treatment of widespread psoriasis.

Tumor necrosis factor (TNF) or TACE inhibitors can be effective against some autoimmune and inflammatory diseases, but thus far disappointing in treating others. While TNF blockers have proven effective in treating rheumatoid arthritis, Crohn's disease, ulcerative colitis, psoriasis, and ankylosing spondylitis, TNF inhibition may interfere with healthy immune mechanisms. Reports of increased infections with M tuberculosis, opportunistic infections, and lymphoma have occurred. Autoimmune diseases have not responded to these drugs, and in some cases have worsened. Despite expectations, TNF blockers have not yet proven effective against multiple sclerosis, sarcoidosis, Sjögren syndrome, and congestive heart failure. Current TNF blockers are generally expensive, although they may help improve a patient's quality of life and, in some cases, may also help avoid surgery. However, TNF blockers also have some disadvantages. For example, they do not work in all diseases, have some undesirable side effects, and can stretch the budget of patients, insurers, and the whole health care system. For example, one study has demonstrated that rheumatoid arthritis patients treated with TNF inhibitors developed methecillin-resistant Staphylococcus aureus (MRSA) infection. In this study, 430 patients were treated with 15 patients developing MRSA. Other illnesses developed in patients included cellulites, sinusitis, mastitis, pneumonia, tuberculosis and sepsis. Recent data also indicates that anti-TNF antibodies treatment of rheumatoid arthritis can cause shingles.

Thus, there still exists a need for treating patients suffering from inflammatory related diseases and disorders.

### SUMMARY OF THE INVENTION

The present invention provides a method for the treatment of a disease or disorder related to an inflammatory condition comprising administering to a patient in need thereof a therapeutically effective amount of a pharmaceutical composition comprising a metal and a thiol. In particular, embodiments of the present invention are directed towards methods of treating inflammatory conditions that are associated with elevated levels of free TNFα. In one embodiment, the present invention can be used to treat and/or ameliorate the effects of a wide variety of diseases or disorders related to an inflammatory condition including one or more of the following inflammation related diseases/disorders: arthritis, rheumatoid arthritis, asthma, psoriasis, systemic lupus erythematosus, inflammatory bowel syndrome, chronic obstructive respiratory diseases (COPD), fibromyalgia and the neurological diseases and disorders multiple sclerosis, Alzheimer's disease, Parkinson's disease, Huntington's disease, vascular dementia, epilepsy, migraines, stroke and trauma.

The anti-inflammatory agent of the present invention comprises a combination of a metal component and thiol component in the form of a mixture or a complex. The inventors of the present invention have surprisingly discovered that metal-thiols can be used to inhibit free TNFα and thereby treat inflammation associated conditions or diseases.

Metal-thiols that can be used in accordance with the present invention include a combination of a Group V metal and a thiol. Suitable Group V metals include bismuth, antimony, arsenic, and combinations thereof. Preferably, the metal comprises bismuth. Thiols that can be used in accordance with present invention include compounds having a thiol group or that contain one or more sulfur atoms capable of existing in the form of sulfhydryl groups under appropriate pH conditions. Bismuth-ethanedithiol (BisEDT) is a particularly preferred metal-thiol that can be used in accordance with the present invention. The metal-thiol compound is typically administered at a dosage level that is from about 2 μg to 200 μg/kg body weight, and more typically from about 10 μg to 40 μg/kg body weight.

In addition to inhibiting free TNFα, metal-thiols have also been shown to increase metal solubility, lower metal toxicity and increases anti-bacterial effects. In addition, treatment with a metal-thiol can also provide a method of suppressing or killing microbial agents and binding with liberating toxins of certain bacteria. The antimicrobial property of the metal-thiol may also be used to suppress microbial growth, reduce microbial load or eliminate microorganisms without releasing toxins or cellular degradation products.

Thus, it can be seen that the claimed invention provides a method of treating inflammatory related diseases and disorders.

### DETAILED DESCRIPTION OF THE INVENTION

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements.

The present invention provides a method of treating inflammation, and in particular, chronic inflammatory diseases, by administering a therapeutically effective dosage of an anti-inflammatory agent comprising a metal-thiol. In particular, the inventors of the present invention have surprisingly discovered that metal-thiols can be used to inhibit free TNFα and thereby treat inflammation associated conditions or diseases.

The present invention can be used to treat and/or ameliorate the effects of a wide variety of diseases or disorders related to an inflammatory condition. In one embodiment, the present invention can be used to treat and/or prevent one or more of the following inflammation related diseases/disorders: arthritis, rheumatoid arthritis, asthma, psoriasis, systemic lupus erythematosus, inflammatory bowel syndrome, chronic obstructive respiratory diseases (COPD), fibromyalgia and the neurological diseases and disorders multiple sclerosis, Alzheimer's disease, Parkinson's disease, Huntington's disease, vascular dementia, epilepsy, migraines, stroke and trauma.

The anti-inflammatory agent of the present invention comprises a combination of a metal component and thiol component in the form of a mixture or a complex. The term “mixture” includes the use of two or more components in sufficiently close proximity to each other that they may interact with each other under conditions of end use for inhibiting free TNFα. Patients in need of treatment for a particular disease are those displaying symptoms or responding to diagnostic tests indicating presence of an inflammatory disease or condition. Patients in need of prophylactic intervention are those who through exposure or otherwise are at higher risk of contracting an inflammatory related disease or condition.

While not wishing to be bound by theory, it is believed that the likely target for metal-thiols at anti-inflammatory levels is the protein metallothionein (MT). MT is a zinc thiol that monitors oxidative changes in the body. The highly sensitive redox nature of thiol:disulfide transitions in MTs regulates immune defenses. MTs also regulate MPs like TACE, by limiting the amount of zinc in blood and tissues. Zinc is an essential co-factor for MPs. Oxidation of MT releases zinc igniting scores of enzymes (i.e., MPs) for growth and repair. These are thiol proteins that are highly sensitive to redox changes in the blood and tissues. The redox component in metal-thiol compounds is the thiol component rather than the metal component, and it may have a direct role in inhibiting zinc release from MT. While not wishing to be bound by theory, it is believed that metal-thiol may interact with Zn in the blood, and exchange cations to create a zinc-thiol bond (e.g., ZnEDT), which returns zinc to MT. Since thiol compounds like glutathione have been shown to stabilize MTs this way, metal-thiol compounds may behave similarly. Without free zinc for enzymes that break down tissue, the effects of inflammation and oxidative damage can be reduced or prevented. At 20-40 μg/kg, metal-thiols may act as a reducing agent to stabilize MT and prevent a cascade of events.

Metal-thiols that can be used in accordance with the present invention include a combination of a Group V metal and a thiol. Suitable Group V metals include bismuth, antimony, arsenic, and combinations thereof. Preferably, the metal comprises bismuth. Thiols that can be used in accordance with present invention include compounds having a thiol group or that contain one or more sulfur atoms capable of existing in the form of sulfhydryl groups under appropriate pH conditions, regardless of whether such sulfur atoms are deprotonated or fully or partially protonated under conditions in which the thiol is used, and regardless of the pH under which it is used. The term “thiol group” includes sulfur or an —SH group of a “thiol”.

Several bismuth, antimony or arsenic salts can be used as the metal component of the invention. Although it is not required that metal be presented in salt form, metal salts can used to put the metal in solution and make it available and accessible to the complexing agents of the invention which include thiol compounds. The preferred salts of the invention are the ones that make the metal more accessible and available to form a complex with the complexing agent. Examples of salts include, but are not limited to, metal nitrate, subgallate, citrate, oxide and subsalicylate. In a preferred embodiment, the metal salt is a bismuth salt such as bismuth nitrate, colloidal bismuth subcitrate and bismuth subsalicylate.

It is not necessary to choose only a single metal or a single thiol for use in the composition of the present invention. A plurality of different metals (e.g., bismuth and antimony together) may be used, as may a plurality of thiols (e.g., ethanedithiol and butane dithiol together). Metals and thiols are presented to the system in a variety of different manners. For example, one metal may be presented as a free ion while the other is added in the form of a salt. Even where only a single metal is used, some of it may be added freely and some may be added in salt form. A wide variety of different salts may be used. Likewise, one or more such variations are permitted when presenting the thiol component into the invention.

In one embodiment, the metal-thiol combination comprises a complex in which the metal component (e.g., bismuth, antimony or arsenic) are chelated by one or more thiol compounds. Metal-thiol complexes are believed to provide enhanced solubility such that the necessary dosage of the metals for effective treatment can be reduced. In one embodiment, for example chelation of the metal component can be achieved by dissolving the thiol compound in a propylene glycol solution of bismuth, antimony or arsenic salt. Thereafter, samples can be further diluted to the desired concentrations using water or propylene glycol.

In a preferred embodiment, the metal-thiol comprises bismuth chelated by a thiol compound containing one sulfhydryl group such as a compound selected from the group consisting of 3-mercapto-2-butanol, β-mercaptoethanol, 2-mercaptoethylamine, monothioglycerol, and p-chlorothiophenol. More preferably, the chelating compound contains a plurality of sulfhydryl groups (for example, two) such as a compound selected from the group consisting of 3,4-dimercaptotoluene, ethanedithiol, 2,3-butanedithiol, 2,3-dimercapto-1-propanol, 1,4-dimercapto-2,3-butanediol, 1,3-propanedithiol, and 1,4-butanedithiol. Bismuth-Ethanedithiol (BisEDT) is particularly preferred.

In one embodiment, the metal-thiol compound is selected from the group consisting of: (A) a mixture comprising (i) a complexing agent having at least one thiol group, and (ii) a group V metal or compound thereof said Group V metal being selected from the group consisting of bismuth, antimony and arsenic; (B) a complex whose molecular structure includes (i) a complexing agent having at least one thiol group, (ii) a Group V metal or compound thereof said Group V metal being selected from the group consisting of bismuth, arsenic and antimony; and (iii) a coordinate bond linking at least one sulfur atom of the thiol-containing complexing agent of subparagraph (B)(i) to the metal of subparagraph (B)(ii); and (C) a combination comprising the complex of paragraph (B) and at least one specie selected from the group consisting of (i) a thiol-containing complexing agent and (ii) a Group V metal or compound thereof, said Group V metal, being selected from the group consisting of bismuth antimony and arsenic.

Either the metal, the thiol, or the complex may be supplied in liquid or solid form. Compositions in accordance with the present invention may further include solvents, diluents, excipients, preservatives, emulsifiers, compounds for adjusting odor, taste, pH or the like.

The formulations can be administered with or without additional carrier or diluent by the oral, systemic injections, percutaneous, transmucosal, or other typical route. Metal-thiol formulations in accordance with the present invention may be administered orally in caplet, tablet, particle, granule, or powder forms. The present invention provides a method of treating and/or ameliorating the effects of inflammation by administering a therapeutically effective amount and/or a prophylactic amount of a metal-thiol formulation or a pharmaceutically acceptable salt thereof, to a sufferer in need thereof. By “therapeutically effective amount” it is meant an amount of the active ingredient (e.g., metal-thiol or a pharmaceutically acceptable salt thereof) to a mammal is effective to treat and/or prevent one or more targeted disorders.

The dosage administered will, of course, vary depending on the use and known factors such as the pharmacodynamic characteristics of the active ingredient; age, health, and weight of the recipient; nature and extent of symptoms, kind of concurrent treatment, frequency of treatment, and the effect desired. The recipient may be any type of mammal, but is preferably a human. In one embodiment, dosage forms (compositions) of the anti-inflammatory metal-thiol formulations may contain about 1 microgram to 100 micrograms of active ingredient per unit, and in particular, from about 10 to 80 micrograms of active ingredient per unit.

For intravenous delivery, a unit dose of the anti-inflammatory metal-thiol formulation will generally contain from 2 to 1,000 micrograms per kg body weight and preferably will contain from 30 to 500 micrograms, in particular 10, 15, 20, 30, 40, 50, 150, 200, or 500 micrograms per kg body weight (μg/kg body weight). The composition may be administered once or more times a day for example 2, 3 or 4 times daily, and the total daily dose for a 70 kg adult will normally be in the range 1.0 to 10 micrograms. Preferred intravenous dosage ranges from 10 ng to 200 μg, preferably 2 to 200 μg, more preferably 10 to 100 μg of metal per kg of body weight. Alternatively the unit dose may contain from 2 to 20 micrograms of metal-thiol and be administered in multiples, if desired, to give the preceding daily dose. In these pharmaceutical compositions, the active ingredient will ordinarily be present in an amount of about 0.5-95% by weight based on the total weight of the composition.

For use in the treatment of targeted inflammatory conditions, by way of general guidance, a daily oral dosage of the active ingredient can generally range from about 0.02 to 10 mg/kg of body weight. In a preferred embodiment, metal-thiol active agent of the invention can be administered at dosages of 0.1 to 4 mg of metal per kg of body weight when administered orally. It should be recognized that the dosage can be raised or lowered based on individual patient response.

As used in the invention, a metal:complexing agent of the invention may be in a pharmaceutical composition for oral administration, a metal:complexing agent is preferably present in a concentration between 5 and 99% by weight relative to total weight of the composition, more preferably between 50 and 99 percent, especially between 80 and 99 percent.

For oral administration when the composition is in the form of a tablet or capsule, the active ingredient can be combined with an oral, non-toxic, pharmaceutically acceptable, inert carrier, including but not limited to, lactose, starch, sucrose, glucose, methyl cellulose, magnesium stearate, dicalcium phosphate, calcium sulfate, mannitol, sorbitol and the like. Additionally, when desired or necessary, suitable binders, lubricants, disintegrating agents, and coloring agents can also be incorporated into the mixture. Suitable binders may include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms may include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

In some embodiments, the metal-thiol formulations of the present invention may also be coupled with soluble polymers as targetable drug carriers. Such polymers can include, for example, polyvinylpyrrolidone, pyran copolymer, polyhydroxypropylmethacrylamide-phenol, polyhydroxyethylaspartamidephenol, or polyethyleneoxide-polylysine substituted with palmitoyl residues. In one embodiment, metal-thiol formulations in accordance with the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

In addition to inhibiting free TNFα, metal-thiols have also been shown to increase metal solubility, lower metal toxicity and increases anti-bacterial effects. In addition, treatment with a metal-thiol can also provide a method of suppressing or killing microbial agents while not liberating toxins or harmful cellular fragments.

The antimicrobial property of the metal-thiol may also be used to suppress microbial growth, reduce microbial load or eliminate microorganisms without releasing toxins or cellular degradation products. Host cell integrity is maintained when the agent is used.

### EXAMPLE 1

In the following examples, the protective effects of BisEDT was studied in an inflammation model of mice. Male swiss white mice weighing between 30-35 grams were used. The cecum of the mice were ligated and perforated to cause inflammation. BisEDT was prepared in a molar concentration of 1:2 by adding 4.9 pi 10.19 M EDT to 1 ml of 50 mM Bi(NO₃)₃ in propylene glycol.

A total of sixty mice, 10 in each group, were used in the survival phase of experimentation. Five hours after CLP, BisEDT was administered intravenously in five groups via the tail vein, once a day for three days, at doses of 0.2, 2, 20, 200 and 2000 ug Bi³⁺/kg, while the last group received vehicle only. The survival time for each group of mice was recorded to determine an optimal dosage of BisEDT. The study was repeated using limited doses to confirm the results, and to determine if more frequent dosing would increase or prolong survival. This group was injected twice daily with the same doses of BisEDT, with one control group.

A total of eight groups each containing 10-20 mice in each group were used for the next phase of the study. Five hours after initiation of inflammation, 20 ug/kg BisEDT was administered intravenously with the combination of different biologics indicated in Table 2 with one group that received vehicle only (control). The survival time for each group was recorded. In another eight groups of mice 10 in each group was injected similarly with the same combination of treatment and control and several measures of immune response were assessed such as measurement of pro- and anti-inflammatory cytokines during BisEDT treatment of these mice. The results show that only 10% of the mice survived in the control group, i.e., mice those did not receive BisEDT (received comparable volumes of the solute). From 60-70% of mice survived when treated with one or two doses of 20 μg/kg BisEDT (p<0.05), whereas doses 10-fold higher or lower protected only 30-40% of mice (Table 1). Mice in the control group showed significantly elevated levels of IL1β, TNFα, IL-10, and reduced levels of TGFβ (p<0.05). In contrast, mice treated with BisEDT showed significant changes in TNFα only, and not nearly to the same extent as in the untreated group (Table 2). However, only free TNFα, not total TNFα or free IL-10, correlated strongly with survival (r=−0.92). IL-10 is an anti-inflammatory cytokine and under certain circumstances it can cause immunosuppression. In order to test whether IL-10 plays a role in the model, monoclonal antibodies to IL-10 (that neutralizes IL-10) were used. The results indicate that antibodies to IL-10 are somewhat protective of mice, but their protective effect was abrogated when combined with BisEDT. Additionally, IL10 or IL-10+BisEDT provided no protection for the mice. A general damping down of cytokine production was observed, yet it was the reduction in free TNFα that correlated most closely with prevention. BisEDT appears to preserve the normal cellular response to activating stimuli and to protect mice from immune suppression or overwhelming immune reaction.

TABLE 1
Survival response to multiple BisEDT dosing

| | % Survival | % Survival |
|---|---|---|
| Dose (μg Bi³⁺/kg BW) | 1 dose | 2 doses |
| saline | 10 | 10 |
| 2 | 40 | 40 |
| 20 | 60* | 70* |
| 200 | 40 | 30 |

After inflammation, BisEDT was introduced into the tail vein once or twice daily for three days. Mice were monitored for 8 days to determine survival. *p<0.05, compared to control.

TABLE 2
Mouse inflammation, intervention, survival and serum cytokines

| | Survived/ | % | tTNFα* | sTNFα* | sIL-10* |
|---|---|---|---|---|---|
| Group | Total | Survived | (ng/ml) | (pg/ml) | (pg/ml) |
| Control | 2/20 | 10 | 1138 | 675 | 1220 |
| BisEDT | 6/10 | 60 | 330 | 100 | 595 |
| Mouse mAb IL-10 | 3/10 | 30 | 1825 | 201 | 525 |
| Mouse mAb | 0/15 | 0 | 670 | 620 | 733 |
| IL-10 + BisEDT | | | | | |
| Human mAb IL-10 | 7/15 | 47 | 436 | 177 | 625 |
| Human mAb | 3/15 | 20 | 397 | 360 | 376 |
| IL-10 + BisEDT | | | | | |
| IL-10 | 0/10 | 0 | — | — | — |
| IL-10 + BisEDT | 0/10 | 0 | — | — | — |

| | | | |
|---|---|---|---|
| Correlation co-efficient | −0.29 | −0.92 | −0.39 |

*t and s refer to total and free fractions, respectively

Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method for treatment of a disease or disorder related to an inflammatory condition comprising administering to a patient in need thereof a therapeutically effective amount of a pharmaceutical composition comprising a metal and a thiol.

2. The method of claim 1, wherein the pharmaceutical composition comprises bismuth-ethanedithiol.

3. The method of claim 2, wherein said bismuth-ethanedithiol is administered intravenously.

4. The method of claim 3, wherein said bismuth-ethanedithiol is administered at a dose of 10 ng to 200 μg/kg of body weight.

5. The method of claim 1, wherein said disease or disorder related to an inflammatory condition is selected from the group consisting of arthritis, rheumatoid arthritis, asthma, psoriasis, systemic lupus erythematosus, inflammatory bowel syndrome and the neurological diseases and disorders multiple sclerosis, Alzheimer's disease, Parkinson's disease, Huntington's disease, vascular dementia, epilepsy, migraines, stroke and trauma.

6. A method of treating patients or preventing chronic inflammatory diseases (CID) in a patient in need thereof comprising administering a therapeutically effective amount of a pharmaceutical composition comprising bismuth-ethanedithiol.

7. The method of claim 6, wherein the inflammatory disease comprises an idiopathic inflammatory bowel disease.

8. The method of claim 6, wherein the inflammatory disease comprises muscular sclerosis and/or psoriasis.

9. The method of claim 6, wherein the inflammatory disease is rheumatoid arthritis.

10. The method of claim 6, wherein the bismuth-ethanedithiol is administered at a dosage from about 2 μg to 200 μg/kg body weight.

11. The method of claim 6, wherein the metal thiol compound is administered at a dosage from about 10 μg to 20 μg/kg body weight.

12. A method of inhibiting or decreasing free TNF in a patient comprising the step of administering a metal-thiol compound selected from the group consisting of:
(A) a mixture comprising (i) a complexing agent having at least one thiol group, and (ii) a group V metal or compound thereof said Group V metal being selected from the group consisting of bismuth, antimony and arsenic;
(B) a complex whose molecular structure includes (i) a complexing agent having at least one thiol group, (ii) a Group V metal or compound thereof said Group V metal being selected from the group consisting of bismuth, arsenic and antimony; and (iii) a coordinate bond linking at least one sulfur atom of the thiol-containing complexing agent of subparagraph (B)(i) to the metal of subparagraph (B)(ii); and
(C) a combination comprising the complex of paragraph (B) and at least one specie selected from the group consisting of (i) a thiol-containing complexing agent and (ii) a Group V metal or compound thereof, said Group V metal, being selected from the group consisting of bismuth antimony and arsenic.

13. The method of claim 12, wherein the metal thiol compound is administered at a dosage from about 2 μg to 200 μg/kg body weight.

14. The method of claim 12, wherein the metal thiol compound is administered at a dosage from about 10 μg to 20 μg/kg body weight.

15. The method of claim 12, wherein the pharmaceutical composition comprises bismuth-ethanedithiol.
